(19) United States
(12) Patent Application Publication
(10) Pub. No.: US 2024/0252549 A1
(43) Pub. Date: Aug. 1, 2024

(54) COMPOSITIONS AND METHODS TO TRANSFECT, TEST, AND TREAT SKIN CELLS

(71) Applicant: The Regents of the University of Michigan, Ann Arbor, MI (US)

(72) Inventors: Michelle J. Kahlenberg, Ann Arbor, MI (US); Mrinal K. Sarkar, Ann Arbor, MI (US); Johann E. Gudjonsson, Ann Arbor, MI (US)

(21) Appl. No.: 18/565,023

(22) PCT Filed: Jun. 16, 2022
(86) PCT No.: PCT/US22/33733

§ 371 Date: Nov. 28, 2023

Related U.S. Application Data
(60) Provisional application No. 63/211,173, filed on Jun. 16, 2021.

(51) Int. Cl. A61K 35/36; A61K 31/713; A61K 45/06; C12N 9/22; C12N 15/11; C12N 15/90

(52) U.S. Cl. CPC A61K 35/36; A61K 31/713; A61K 45/06; C12N 9/22; C12N 15/11; C12N 15/907; C12N 2310/20

(57) ABSTRACT

The present invention provides compositions and methods for enhancing transfection efficiency. In particular, the present invention provides compositions and methods that increase transfection efficiency of skin cells, including keratinocytes, which facilitates use of gene expression modifying technologies such as CRISPR-Cas technologies.

### CROSS-REFERENCE TO RELATED APPLICATION

The present application claims priority to U.S. Provisional Patent Application No. 63/211,173, filed Jun. 16, 2021, which is hereby incorporated by reference in its entirety.

### FIELD

The present invention provides compositions and methods for enhancing transfection efficiency. In particular, the present invention provides compositions and methods that increase transfection efficiency of skin cells, including keratinocytes, which facilitates use of gene expression modifying technologies such as CRISPR-Cas technologies.

### BACKGROUND

There are hundreds of genetically based skin disorders, including at least dozens that are single gene mutation based. Many are debilitating and significantly hinder quality of life. A direct approach to study and treat these conditions would be to use available tools to modify genetic expression of these mutations. Skin cells, however, have proved highly resistant to transfection. Keratinocytes (KCs), which make up 90% of skin cells, in particular, have shown extreme resistance to transfection in comparison to other cell types. The low level of transfection has limited the gene therapy applications for skin conditions, diseases, and disorders. There are many possible applications of gene therapy, or other treatments, that could moderate cell protein expression that could counter subsets of genetic mutation-based diseases and disorders, if efficient transfection of skin cells were available. Researchers have shown that KCs are candidates for genetic based treatments through transfection and use of gene therapy tools, including CRISPR-Cas technologies. A key challenge to further exploration and development of these gene therapy and related approaches has been the difficulty in transfecting skin cells, especially KCs. KCs have long been recognized as one of the most difficult cell types to transfect (12, 13), but the mechanisms behind this resistance have remained unknown. KCs are the major cellular constituent of the epidermis that has a critical role and acts as the primary interface between our body and external agents, such as bacteria and viruses.

### SUMMARY

The present invention provides compositions and methods for enhancing transfection efficiency. In particular, the present invention provides compositions and methods that increase transfection efficiency of skin cells, including keratinocytes, which facilitates use of gene expression modifying technologies such as CRISPR-Cas technologies.

In some embodiments, provided herein are methods comprising: contacting skin cells with one or more agents that regulate interferon kappa (IFNκ) expression in the skin cells to enhance transfection of the skin cells with one or more nucleic acid molecules. In some embodiments, the one or more agents comprise a JAK inhibitor (e.g., a JAK1 and/or JAK2 inhibitor). In some embodiments, following or concurrently with the agent contact, the cells are contacted with the one or more nucleic acid molecules to transfect the cells. In some embodiments, the nucleic acid molecules are complexed with one or more transfection reagents. Transfection reagents and systems include, but are not limited to, calcium phosphate, electroporation systems, lipofection system, microinjection systems, gene guns, impalefection systems, hydrostatic pressure systems, and the like. Transfection may be stable or transient. Transfected nucleic acid may be DNA or RNA or combinations thereof.

In some embodiments the one or more nucleic acid molecules to be transfected into the skin cells comprise one or more components of a CRISPR-Cas system. For example, in some embodiments, one or more vectors is transfected that encodes one or more components of a CRISPR-Cas system (e.g., a guide sequence, one or more protein components of the CRISPR-Cas system, a cargo sequence to be integrated into a genome or other nucleic acid present in the skin cell, etc.). In some embodiments, one or more components of a CRISPR-Cas system is provided as a protein or protein complex.

In some embodiments, the one or more nucleic acid molecules comprise components useful for, necessary for, of sufficient for gene therapy or genetic manipulation of a skin cell. For example, one or more transgenes may be provided to the skin cell in a manner that permits expression of the transgene in the skin cell. The transgene may be integrated into the skin cell genome or may be expressed episomally.

The invention is not limited by the nature of the mechanism of transgene delivery or nucleic acid editing. Such mechanisms include, but are not limited to, naked nucleic acid, CRISPR-Cas, Cre-lox, viral system delivery (e.g., retrovirus, adenovirus, herpes simplex, vaccinia, adeno-associated virus), electroporation, gene gun system, sonoporation, magnetofection, use of lipoplexes, use of dendrimers, use of inorganic nanoparticles, and the like.

In some embodiments, the nucleic acid delivered is a transgene, a mRNA, an siRNA, an antisense oligonucleotide, a guide RNA, or the like. A transgene may encode a regulatory molecule (e.g., regulatory protein), a therapeutic molecule, a diagnostic biomarker, or the like. An inserted genetic sequence may provide a regulatory sequence rather than encoding a molecule. For example, the inserted genetic sequence may provide a regulatory sequence (e.g., a promoter, enhancer, etc.) or provide a detectable biomarker (e.g., a unique barcode), a landing pad for facilitating further genetic manipulation, a cleavage site, information content (e.g., for genetic storage of data), or the like.

In some embodiments, a transgene is added to the cells where the transgene is involved in regulating a skin cell disease or condition. Skin diseases and conditions include, but are not limited to, acne, cold sores, blisters, hives, actinic keratosis, rosacea, carbuncles, allergies, eczema, psoriasis, cellulitis, measles, cancers (e.g., basal cell carcinoma, squamous cell carcinoma, melanoma), lupus, contact dermatitis, vitiligo, warts, chickenpox, seborrheic eczema, keratosis pilaris, ringworm, melasma, impetigo, wounds, infections (e.g., bacterial, viral, fungal), moles, candidiasis, athlete's foot, dermatomyositis, shingles, age spots, and the like. In some embodiments, symptoms include any one or more of raised bumps, rash, itchiness, scaliness, peeling, ulcers, open sores or lesions, dryness, cracking, discoloration, and loss of pigment, flushing. The technology finds use in research, therapeutic, and diagnostic methodologies.

In some embodiments, the transfection is used to correct or counteract a genetic anomaly responsible for a disease or condition. A wide variety of genetic profiles have been associated with skin disease (see e.g., DeStefano and Christiano, Cold Spring Harb. Perspect. Med. 4(10), 2014, “The Genetics of Human Skin Disease”; Sybert, “Genetic Skin Disorders”, 3 ed., Oxford University Press, 2017; Shinkuma, “Advances in Gene Therapy and their Application to Skin Diseases: A review”, J. Dermatol. Sci., S0923-1811, May 21, 2021); herein incorporated by reference in their entireties).

In some embodiments, the transfection is used for research purposes.

The invention is not limited by the nature of or the location of the skin cells. In some embodiments, the cells are keratinocytes. In some embodiments, the skin cells are melanocytes, Langerhans cells, or Merkel cells. In some embodiments, the cells are in vitro (e.g., in culture). In some embodiments, the cells are ex vivo. In some embodiments, the cells are in vivo. The cells may comprise a mixture of different types of skin cells and may comprises other cell types present in tissues containing skin cells (e.g., mast cells, vascular smooth muscle cells, fibroblasts, immune cells, neutrophils, T and B Lymphocytes, eosinophils, monocytes, and the like). The skin cells may be present in natural or synthetic skin tissues. The skin cells may be differentiated, cultured, harvested, printed, or otherwise collected or generated in any desired manner. The skin cells may be of human origin, or may be from other organism including, but not limited to, companion animals (e.g., dogs, cats, etc.), livestock (e.g., cattle, pigs, chickens, etc.), wildlife animals (e.g., lions, tigers, bears, dolphins, whales, wolves, etc.), mammals, birds, fish, horses, and the like.

Also provided herein are compositions of matter that are useful for, sufficient for, or necessary for the practice of the methods described herein. Compositions include, but are not limited to, kits (collections of materials packaged together in one or more containers and designed for use together), reactions mixtures (collections of materials combined together to perform one or more reactions), reagents, and systems (two or more components designed to work together, including, but not limited to reagents, cells, instruments, software, and the like). Compositions may include control reagents (e.g., positive and/or negative control reagents), which can include control cells, control transfection reagents, control nucleic acid molecules, and the like.

In some embodiments, the compositions comprise: one or more agents (e.g., JAK1 and/or JAK2 inhibitors), one or more transfection reagents, and one or more nucleic acid molecules to be transfected. In some embodiments, the one or more nucleic acid molecules comprises a vector. In some embodiments, the vector expresses one or more components of a CRISPR-Cas system. In some embodiments, the one or more nucleic acid molecules comprises a guide sequence. In some embodiments, the vector expresses one or more transgenes. In some embodiments, the composition further comprises one or more skin cells (e.g., keratinocytes).

JAK (Janus kinase) inhibitors include JAK1 and JAK2 inhibitors as well as inhibitors of other members of the Janus kinase family of enzymes (e.g., JAK3, TYK2). Such inhibitor include, but are not limited to, Ruxolitinib (trade names Jakafi/Jakavi) (JAK1/JAK2), Tofacitinib (trade names Xeljanz/Jakvinus, formerly known as tasocitinib and CP-690550) (JAK3), Oclacitinib (trade name Apoquel) (JAK1), Baricitinib (trade name Olumiant) (JAK1/JAK2), Peficitinib (ASP015K, JNJ-54781532; trade name Smyraf) (JAK3), Fedratinib (SAR302503; trade name Inrebic) (JAK2), Upadacitinib (trade name Rinvoq; ABT-494) (JAK1), Filgotinib (G-146034, GLPG-0634) (JAK1), Cerdulatinib (PRT062070) (dual SYK/JAK), Gandotinib (LY-2784544) (JAK2), Lestaurtinib (CEP-701) (JAK2), Momelotinib (GS-0387, CYT-387) (JAK1/JAK2), Pacritinib (SB1518) (JAK2), Abrocitinib (PF-04965842) (JAK1), Deucravacitinib (BMS-986165) (TYK2), Ruxolitinib cream (INCB018424) (JAK1/2), Cucurbitacin I (JSI-124), CHZ868 (JAK2), Tofacitinib, and Topical tofacitinib and ruxolitinib, as well as compounds described in U.S. Pat. Nos. 8,609,687, 8,637,526, 8,987,443, 9,422,300, 9,890,165, 10,023,577, 10,100,049, 10,435,428, 10,493,077, 10,617,690, 10,758,543, 10,786,507, and 10,968,222 and US Pat. Apln. Ser. Nos. 20210155621, 20210030672, and 20200190080, each of which is herein incorporated by reference in its entirety.

In some embodiments, provided here are uses of any of the above compositions are compositions described elsewhere herein. For example, in some embodiments, provided herein are uses of such compositions for transfecting a skin cell. In some embodiments, provided herein are uses of such compositions to study, prevent, or treat a disease or condition.

### BRIEF DESCRIPTION OF THE DRAWING(S)

FIG. 1A-G. Keratinocyte activate type I IFN responses through the STING pathway and are resistant to CRISPR-cas9 transfection (A) Comparison of transfection efficiency in keratinocytes, fibroblasts and human embryonic kidney 293T (HEK-293T) cells (n=3; unpaired t test; ***P<0.001; mean±SEM). (B) IFNK and MX1 mRNA expression in keratinocytes, fibroblasts and HEK-293T cells (n=3; unpaired t-test; ***P<0.001; mean±SEM). (C) Induction of mRNA expression of the type I IFN; IFNK, and IFN response gene MX1 by CRISPR plasmid (n=3; unpaired t-test; **P<0.01, ***P<0.001; mean±SEM). (D) IFNK and MX1 expression in CRISPR plasmid treated WT and TMEM173 (STING) KO keratinocytes (n=3; unpaired t-test; *P<0.05, **P<0.01, ***P<0.001; mean±SEM). Bars with blue dots: no treatment; bars with red dots: CRISPR plasmid treatment (E) Phospo-IRF3 western blot in plasmid treated KO keratinocytes. (F) Single-cell ATAC-seq from healthy human epidermis shows overlap between IFNK, MX1 and KRT5 open chromatin regions (upper panel). Chromatin accessibility in the IFNK promoter region is greater in undifferentiated keratinocytes compared to differentiated keratinocytes. (G) Heatmap of type I IFN responsive genes from scRNA-seq data of healthy human epidermis shows localization of majority of IFN response genes in the basal epidermal compartment (n=3).

FIG. 2A-F. STING dependent induction of the cytidine deaminase APOBEC3G restricts CRISPR/Cas9 transfection efficiency in keratinocytes. (A) Percentage of GFP positive cells at different time points after CRISPR transfection. (n=3; unpaired t-test; *P<0.05, **P<0.01, ***P<0.001; mean±SEM). (B) APOBEC3s mRNA expression in IFN-α treated keratinocytes and IFNK KO keratinocytes (n=3; unpaired t-test; *P<0.05, **P<0.01, ***P<0.001; mean f SEM) (C) CRISPR plasmid stability in APOBEC3s siRNA treated keratinocytes (n=3; unpaired t-test; ***P<0.001; mean±SEM). (D) Expression of IFNK, APOBEC3G and FLG mRNA in subconfluent monolayer cultures and 3D epithelial raft cultures at different stages of differentiation (day(D)3 through D12) (n=3; unpaired t-test; *P<0.05, **P<0.01, ***P<0.001; mean±SEM). (E) APOBEC3G and IFN-k immunostaining in healthy skin (n=3; APOBEC3G (red), IFN-k (green)). (F) APOBEC3s mRNA expression in TMEM173 KO keratinocytes (n=3; unpaired t-test; *P<0.05, **P<0.01, ***P<0.001; mean±SEM).

FIG. 3A-F. CRISPR-cas9 generated keratinocytes KOs have suppressed type I IFN responses and IFNK expression. (A) Decreased IFNK expression and type I interferon response (MX1 expression) in CRISPR-cas9 generated KO keratinocytes (n=3; unpaired t-test; ***P<0.001; mean±SEM). (B) Reversal of IFNK expression in CRISPR-cas9 generated KO keratinocytes after treatment with the demethylating agent 5-dAza-c (n=3; unpaired t-test; **P<0.01, ***P<0.001; mean±SEM). (C) CpG hypermethylation in the IFNK promoter region in KO keratinocytes (KO #1 and KO #2) compared to non-transfected WT control (n=8). (D) Western blot of the DNA methyltransferase, DNMT3B in transgenic overexpressing DNMT1, DNMT3A, and DNMT3B (OE) keratinocytes (each lane is representative of n=3 independently transfected KCs (upper panel)). Suppression of IFNK and APOBEC3G mRNA expression in DNMT3B transgenic keratinocytes (lower panel, n=3; unpaired t-test; **P<0.01, ***P<0.001; mean±SEM). (E) DNMT3B mRNA expression in 3D epithelial rafts at different stages of differentiation (n=3; unpaired t-test; *P<0.05, **P<0.01, ***P<0.001; mean±SEM). (F) DNMT3B protein expression is low in basal layer (arrows) but increases progressively in the more differentiated layers of the epidermis (n=3).

FIG. 4A-F. JAK1/JAK2 inhibition prevents suppression of type I IFN response in CRISPR-cas9 generated KO keratinocytes. (A) Increased transfection efficiency in CRISPR-cas9 generated KO keratinocytes (control), and keratinocytes with KO of either IFNK or TYK2 (n=3; unpaired t-test; ***P<0.001; mean±SEM). (B) CRISPR-cas9 generated KO (IFNK KO) keratinocytes, have increased CRISPR-cas9 plasmid stability (n=3; unpaired t-test; *P<0.05, **P<0.01, ***P<0.001; mean±SEM). (C) Suppression of IFNK and MX1 mRNA expression in baricitinib (JAK1/JAK2 inhibitor) treated keratinocytes (n=3; unpaired t-test; **P<0.01, ***P<0.001; mean±SEM). (D) CRISPR/Cas9 transfection efficiency in baricitinib treated keratinocytes (n=3; unpaired t-test; ***P<0.001; mean±SEM). (E) IFNK and MX1 mRNA expression in the CRISPR-cas9 generated KO keratinocytes with (w) or without (wo) JAK1/JAK2 inhibitor (JAKi, n=3; unpaired t-test; **P<0.01, ***P<0.001; mean±SEM). (F) CpG methylation in the IFNK promoter region in JAK1/JAK2 inhibitor treated CRISPR KO keratinocytes. (n=8).

FIG. 5. IFNB1 expression in CRISPR plasmid treated keratinocytes. IFNB1 expression in CRISPR plasmid treated WT and TMEM173 (STING) KO keratinocytes (n=3; unpaired t-test; ***P<0.001; mean±SEM). Bars with blue dots: no treatment; bars with red dots: CRISPR plasmid treatment.

FIG. 6. Chromatogram and western blot for TMEM173 KO keratinocytes. TMEM173 (STING protein) KO keratinocytes were generated by CRISPR-Cas9. Chromatogram shows homozygous mutation with 7 nucleotides deletion. STING western blot in TMEM173 KO KCs.

FIG. 7. MX1 expression in CRISPR-cas9 generated KO keratinocytes after treatment with the demethylating agent 5-dAza-c. (n=3; unpaired t-test; ***P<0.001; mean±SEM).

FIG. 8. CpG methylation analysis in DNMT3B overexpressed keratinocytes. CpG hypermethylation in the IFNK promoter region in the DNMT3B overexpressed compared to control overexpressed keratinocytes (n=3).

FIG. 9. DNMT3B expression in CRISPR KO keratinocytes. (n=3; unpaired t-test; ***P<0.001; mean±SEM).

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

The terms “overexpress” and “overexpression,” as used herein, refer to the expression of a gene beyond normal (or wild-type) levels, or to expression of a gene in a cell type or developmental stage or condition in which it normally is not expressed. Overexpression is also referred to in the art as “misexpression” and “ectopic expression.” A gene is overexpressed if the expression is increased by at least about 20% (e.g., 25%, 30%, 35%, 40%, 45%, 50%, 55%, 60%, 65%, 70%, 75%, 80%, 85%, 90%, 95%, 99%, 100%, 200%, 300%, 500%, or more) as compared to a reference level, control, or wild-type expression level. Levels of expression can be determined according to any of many acceptable protocols known in the art that measure the abundance of encoding RNA (e.g., mRNA), such as quantitative or semi-quantitative polymerase chain reaction (PCR) or northern blot. In other embodiments applicable to protein-coding genes, the expression can be quantified in terms of amount of target protein detected, such as by western blot.

As used herein, the term “antibody” refers to a whole antibody molecule or a fragment thereof (e.g., fragments such as scFv, Fab, Fab′, and F(ab′)2), unless specified otherwise; an antibody may be a polyclonal or monoclonal antibody, a chimeric antibody, a humanized antibody, a human antibody, etc. In a native antibody, a heavy chain comprises a variable region, VH, and three constant regions, CH1, CH2, and CH3. The VH domain is at the amino-terminus of the heavy chain, and the CH3 domain is at the carboxy-terminus. In a native antibody, a light chain comprises a variable region, VL, and a constant region, CL. The variable region of the light chain is at the amino-terminus of the light chain. In a native antibody, the variable regions of each light/heavy chain pair typically form the antigen binding site. The constant regions are typically responsible for effector function.

As used herein, the terms “administration” and “administering” refer to the act of giving a drug, prodrug, therapeutic, or other agent to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein the term “transfection efficiency” refers to, for example, the percentage of target cells, within a population of target cells, that contain an introduced exogenous nucleic acid molecule. Transfection efficiency can be determined by transfecting a nucleic acid molecule encoding a reporter gene into a population of target cells and determining the percentage of cells having reporter activity. The term “transfection efficiency” also refers to the amount of gene product detected following transfection of the nucleic acid into the cell. This is determined, for example, by testing an entire cell population for the amount of gene product produced after a given incubation period. Thus, the term “transfection efficiency” involves assaying for the relative expression of the gene product encoded by the introduced nucleic acid.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention (e.g., testing such a compound on a cell modified by a composition or method described herein). A “known therapeutic compound” refers to a therapeutic compound that has been shown (e.g., through animal trials or prior experience with administration to humans) to be effective in such treatment or prevention.

The term “effective amount” as used herein means that amount of active compound or pharmaceutical agent that elicits the biological or medicinal response in a cell, tissue, organ, system, animal, or human that is being sought by a researcher, veterinarian, medical doctor, or other clinician. In some embodiments, the effective amount is a “therapeutically effective amount” for the alleviation of the symptoms of the disease or condition being treated. In some embodiments, the effective amount is a “prophylactically effective amount” for prophylaxis of the symptoms of the disease or condition being prevented. The term also includes herein the amount of an agent sufficient to directly or indirectly inhibit a JAK kinase or IFNκ and thereby elicit a response being sought (e.g., an “inhibition effective amount”). In some embodiments, this amount is between 0.1 mg and 1000 mg per day, e.g., between 1 mg and 500 mg per day (between 1 mg and 200 mg per day), although the amounts may vary depending on the location and form of the cell being treated (e.g., in vitro, in vivo). Nucleic acid doses, provided by vector, for gene therapy, may be referred to by numbers of vector genomes per kilogram bodyweight (vg/kg) and may vary, for example, from 1×10¹² to 1×10¹⁵.

### DETAILED DESCRIPTION

The disclosure is predicated, at least in part, on the discovery that regulation of IFNκ (e.g., inhibition by contacting cells with a JAK1 and/or JAK2 inhibitor) makes skin cells (e.g., keratinocytes) more amenable to transfection and increases efficiency of transfection. Thus, provided herein are compositions and methods that facilitate transfection of skin cells that have historically been notoriously challenging to transfect.

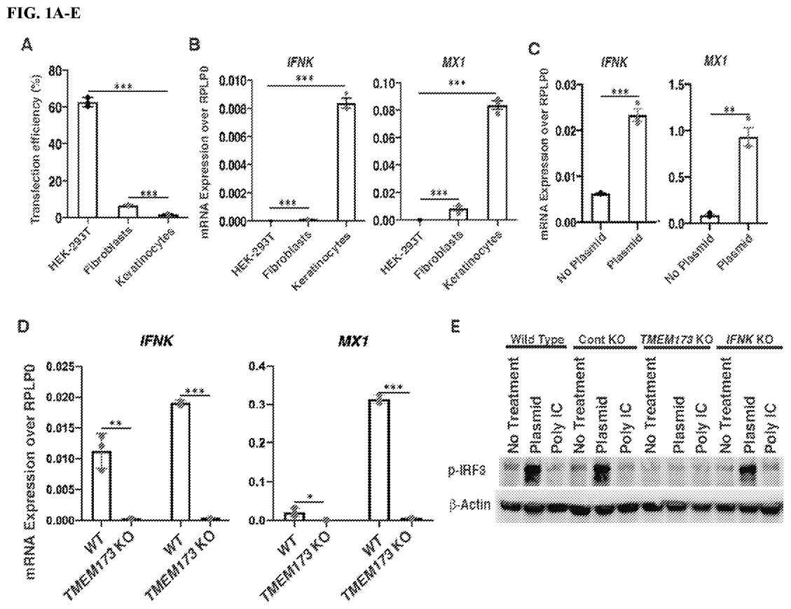

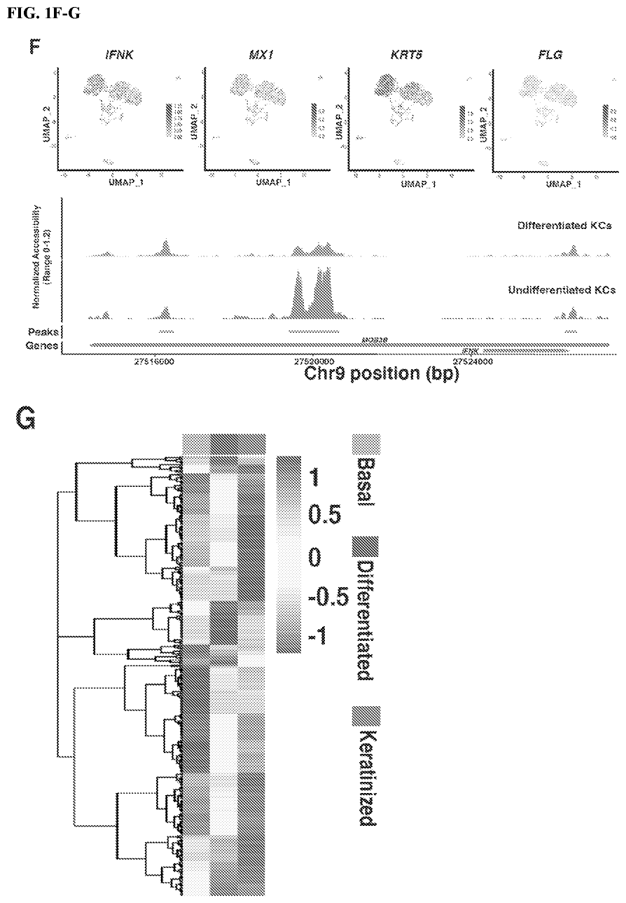

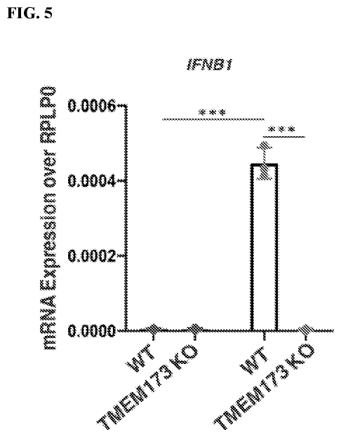

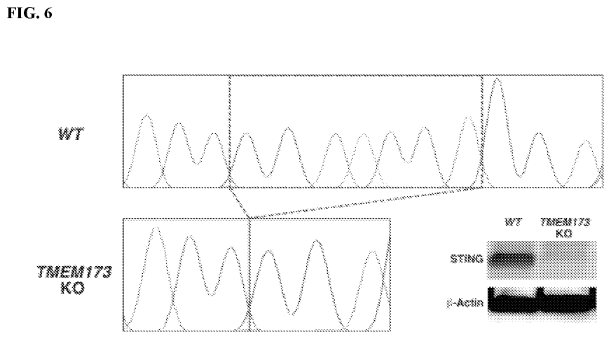

To determine transfection resistance of keratinocytes (KCs), transfection efficiency was compared in human embryonic kidney-239T (HEK293T) cells, fibroblasts, and KCs. Transfection efficiency using liposome-based system, was greater than 60% in HEK-293T cells, compared to 7% in fibroblasts, and only 1% in keratinocytes (KCs) (FIG. 1A). The mechanism behind this resistance of KCs to transfection has been unclear. It was observed that KCs have constitutive expression of the interferon-stimulated gene (ISG) MX1, whereas this was seen in neither fibroblasts nor HEK-293T cells. This corresponded to increased expression of the type I IFN, IFNκ, which was only found in KCs, and not fibroblasts or HEK-293T cells (FIG. 1B). Notably, a significant increase in both IFNK (interchangeably referred to as IFNκ and IFN-κ herein) and MX1 mRNA expression was observed in KCs after CRISPR plasmid transfection (FIG. 1C), suggesting that CRISPR plasmid is recognized by intracellular nucleotide sensors in KCs. Induction of IFNB1 mRNA expression was also observed following CRISPR transfection in KCs but approximately 30-fold less than that of IFNK (FIG. 5). Stimulator of interferon genes (STING) pathway is known to control the induction of innate immune genes in response to the recognition of double-stranded DNA (dsDNA) (15, 81). To address the role of STING in response to CRISPR transfection in KCs, TMEM173 (STING protein) knock out (KO) in KCs (FIG. 6) were generated. In contrast to WT KCs, TMEM173/STING KO completely abrogated both IFNK and MX1 mRNA expression and the IFN response to CRISPR transfection (FIG. 1D). STING activation results in recruitment of the transcription factor, interferon regulatory factor 3 (IRF3) and promotes phosphorylation of IRF316 to activate type I IFNs and ISGs. Phosphorylation of IRF3 (p-IRF3) was assessed by western blot in WT and KO KCs including TMEM173 and IFNK KOs. Whereas robust p-IRF3 was seen in WT, control KO, and IFNK KO KCs, p-IRF3 was markedly reduced in the TMEM173 KO KCs upon CRISPR-Cas9 transfection (FIG. 1E). These data suggest that CRISPR-Cas9 transfection induces IFNκ, and ISGs, in KCs through activation of the STING pathway. Notably, this activation of the STING pathway was not dependent upon constitutive activity of IFN-κ. IFN-κ is a poorly studied member of the type I IFN family but has an established role for host defense against viral infection such as human papilloma viruses (HPV) (17,18). Interestingly, HPV infections typically do not involve the basal layer of the epidermis and are instead localized in the upper spinous layers (19). Indeed, both TMEM173/STING and IFNK mRNA expression was highest in undifferentiated KRT5+96 basal epithelium, in contrast to more differentiated KCs (FLG) and corresponded to open chromatin areas around the IFNK promoter as shown by single-cell ATAC-seq (FIG. 1F). Consistent with this observation, single cell RNA-sequencing of epidermal cells demonstrated that both IFNκ and majority of ISGs are primarily expressed in the basal layer of the epidermis (FIG. 1G). These observations suggest that KCs in the basal layer of the epidermis are more resistant to CRISPR-Cas9 transfection.

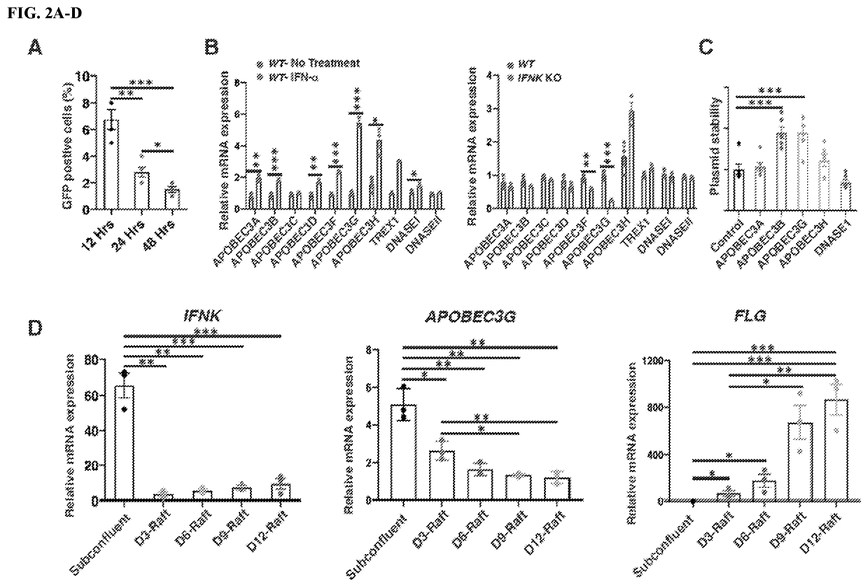

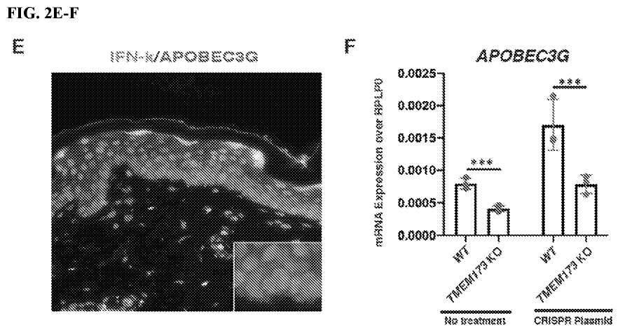

To determine if uptake of the CRISPR-Cas9 plasmid is defective in keratinocyte the uptake and stability of CRISPR-Cas9 GFP-tagged plasmids in KCs was measured at different timepoints after transfection. While CRISPR-Cas9 GFP+ was observed in approximately 6-8% of KCs at early timepoints this rapidly decreased over a period of 48 hours down to 1-2% (FIG. 2A). This uptake followed by rapid disappearance suggest that KCs actively degrade the CRISPR plasmid shortly after transfection, and prior to interaction of CRISPR-Cas9 with its DNA target. DNAses such as DNAse I and DNAse IL, along with the APOBEC3 protein family of cytidine deaminases, have been shown to mediate clearance of foreign DNA from human cells (20-22). To determine involvement of DNASE I, DNASE IL, and APOBEC3 family members in the clearance of CRISPR-Cas9 plasmids from KCs after transfection, RNA-seq was used to compare the expression profiles for type I IFN treated versus IFNK KO KCs. While the majority of the APOBEC3 family members showed increased mRNA expression, only minor shifts were seen for DNASE1 and no changes were observed for DNASE2 mRNA expression. Correspondingly, IFNK KO KCs had decreased mRNA expression of three of the APOBEC3 members; APOBEC3A, APOBEC3F and APOBEC3G, whereas only APOBEC3H was increased (FIG. 2B). To determine the potential role of these four APOBEC3 members and DNASE1 in CRISPR-Cas9 plasmid stability, an siRNA approach was used to knock-down each of the four APOBEC3 genes and DNASE1. Of these five, observed increased plasmid stability in the siAPOBEC3B and siAPOBEC3G KCs was observed (FIG. 2C). To determine relationship of APOBEC3 with epidermal differentiation and IFNK mRNA expression RNA-seq data was analyzed from monolayer KCs and epidermal raft systems. This showed inverse relationship with differentiation stage of both IFNK and APOBEC3G, with more differentiated KCs having lower expression (FIG. 2D). Consistent with these data, APOBEC3G was primarily expressed in the basal layer of skin epidermis co-localizing with IFNκ (FIG. 2E). Consistent with the role of TMEM173/STING in regulating IFN responses to CRISPR-Cas9 transfection, significant suppression of APOBEC3G mRNA expression in TMEM173 KO KCs was observed (FIG. 2F). These data suggest that STING/IFNκ dependent induction of APOBEC3 cytidine deaminases are responsible for CRISPR-Cas9 plasmid degradation in KCs.

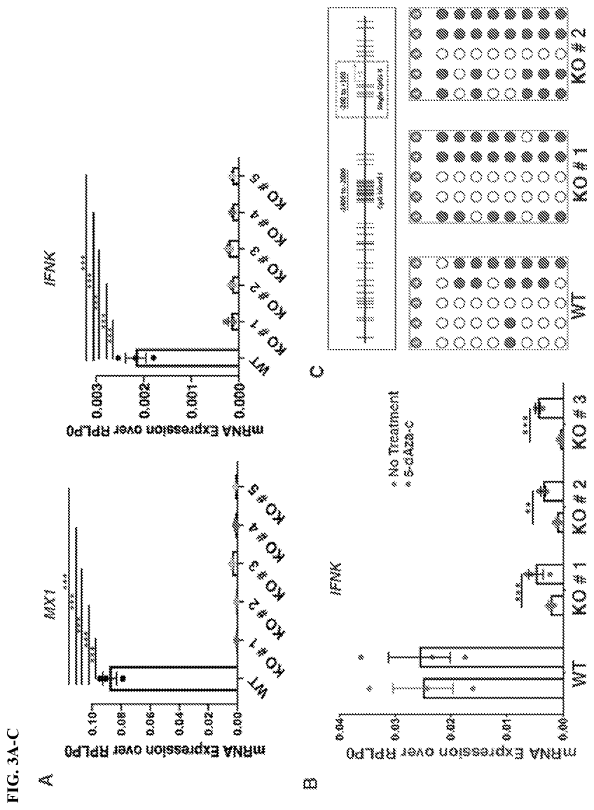

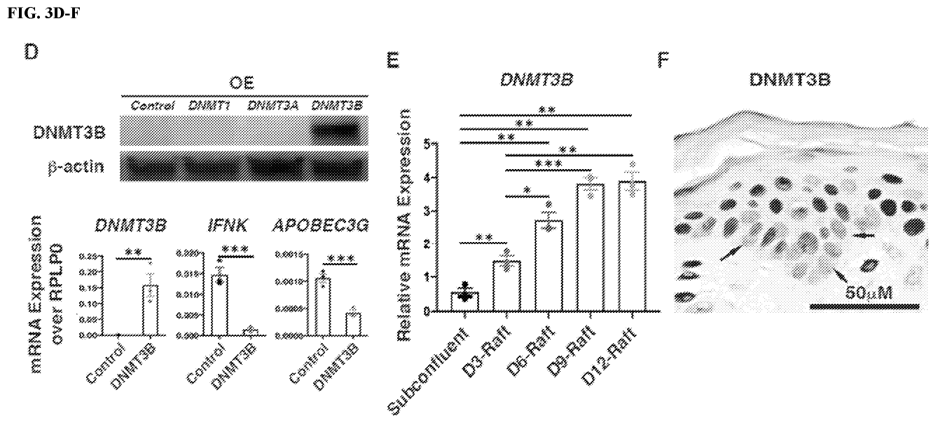

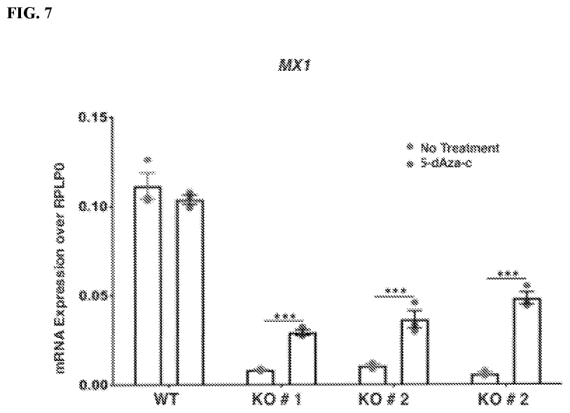

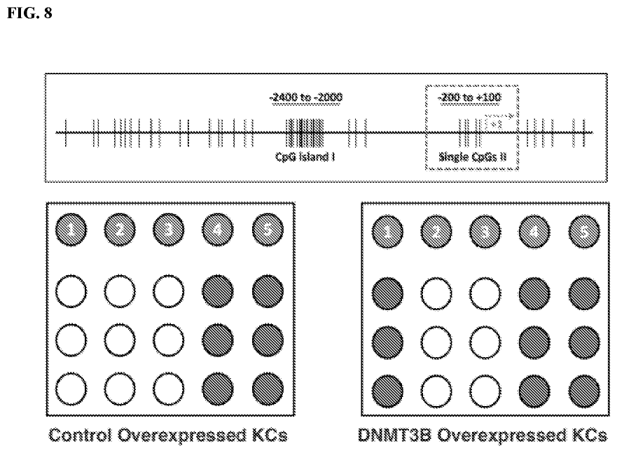

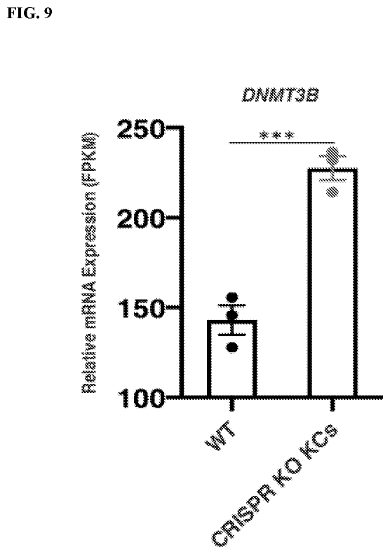

Surprisingly, it was observed that CRISPR/Cas9 generated KOs in KCs have suppressed IFNK and ISG mRNA expression, and this was consistent across all KC KOs generated, irrespective of the gene target (FIG. 3A). CpG methylation is a common epigenetic mark for transcriptional regulation (23), and to determine if this mechanism could explain this suppressed of IFNK and ISGs KO KCs were treated with a demethylating agent, 5-dAza-c. This led to significant increase of both IFNK (FIG. 3B) and the ISG MX1 mRNA expression (FIG. 7) in all KO KCs treated. To determine if CpG methylation was responsible for suppression of IFNK mRNA expression CpG methylation in the IFNK promoter region was analyzed using bisulfite sequencing. There was marked increase in CpG methylation in the CRISPR KO compared to WT KCs (FIG. 3C). DNA methyltransferases (DNMTs) are involved in the CpG methylation (24), and are expressed in skin (25). To determine the role of DNMT methyltransferases in IFNκ regulation, DNMT1, DNMT3A and DNMT3B overexpressing KCs were generated. Only DNMT3B overexpression led to significant suppression of IFNK mRNA expression (FIG. 3D) and this was accompanied by CpG hypermethylation of the IFNK promoter region (FIG. 8). DNMT3B expression positively correlates with epidermal differentiation; indeed, it is most highly expressed in fully differentiated epidermal rafts (FIG. 3E) and inversely correlates with IFNK mRNA expression (FIG. 2D). Consistent with these findings, DNMT3B expression was higher in KO compared to WT KCs (FIG. 9). Confirmatory immunostaining in healthy epidermis showed preferential nuclear expression of the DNMT3B protein in the upper layers of the epidermis whereas there was minimal staining in lower layers of the epidermis (FIG. 3F), where IFNκ and APOBEC3G staining was most robust (FIG. 2E). These data demonstrate link between DNMT3B, IFNκ expression, and epidermal differentiation and provide novel insights into the mechanisms that regulate IFNκ activity in the epidermis.

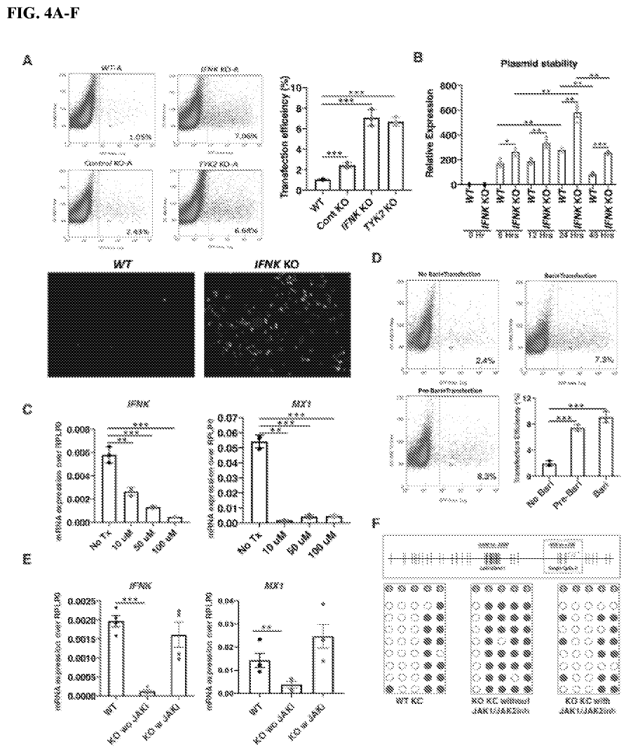

Keratinocyte expression of IFNκ is induced by CRISPR-Cas9 transfection, and IFNκ directly affects expression of APOBEC3 cytidine deaminases that in turn promote degradation of intracellular CRISPR-Cas9 plasmids. To determine if inhibiting type I IFN signaling affects CRISPR-Cas9 transfection efficacy IFNK and TYK2 KO KCs were used. Thus, a marked increase in transfection efficiency (indicated by increased GFP positivity) in both IFNK and TYK2 KO KCs was observed. Furthermore, the control KO KCs had increased transfection efficacy compared to WT KCs (FIG. 4A), likely due to suppressed IFNκ autocrine responses (FIG. 3A). Consistent with these findings, increased stability of CRISPR plasmid over time was observed in the IFNK KO KCs (FIG. 4B). To validate these findings and determine if pharmacologic inhibition of Janus kinase (JAK)/IFN signaling would reproduce these findings, the JAK1/JAK2 inhibitor, baricitinib, was used. Baricitinib effectively decreased mRNA expression of both IFNK and the ISG MX1 in a dose-dependent manner (FIG. 4C), and increased transfection efficiency (FIG. 4D) to the same level as seen in either IFNK or TYK2 KO KCs (FIG. 4A). To determine whether IFNκ affects and promotes selection of IFNK and ISG low expressing KC KOs, CRISPR-Cas9 transfection was performed in the presence or absence of baricitinib. Notably, KC KOs generated in the presence of baricitinib had intact IFNK and MX1 expression, whereas IFNK and MX1 expression was suppressed in KOs in the absence of baricitinib (FIG. 4C), similar to what had previously been observed (FIG. 3A). KC KOs generated in the presence of baricitinib did not have hypermethylation of the IFNK promoter region, in stark contrast to KC KOs generated without baricitinib (FIG. 4F).

KCs constitute ˜90% of the cells in the epidermis (26). Given the constant onslaught of external agents and microbiota such as bacteria and viruses, KCs are highly active as a sentinel cells harboring a range of antimicrobial detectors and pattern recognition receptor for a wide range of viruses and bacteria (27). IFNκ is the predominant type I IFN expressed by KCs and is most prominently expressed in the basal layer of the epidermis (28). The role of this axis in anti-viral defenses can be best described in the context of human HPV infections, which are caused by a DNA virus. HPV infections classically involve the mid to upper layers of the epidermis (29), where HPV viral genome amplification occurs (30). Interestingly, HPV viruses antagonize the cGAS-STING-DNA-sensing pathway to facilitate infection (31). Here it is demonstrated that CRISPR, which is constituted out of a DNA segment containing short repetitions of bases sequences, activates the same type of anti-viral response through STING and identify cytidine deaminase APOBEC3G as a key regulator in controlling CRISPR transfection in KCs (FIG. 2B-C).

A surprising observation was that CRISPR KC KOs had permanent suppression of IFNκ mRNA expression and ISG response through IFNK promoter hypermethylation. Under normal physiologic conditions in the epidermis IFNκ expression shows a sharp cutoff in expression once KCs leave the basal layer of the epidermis (28), suggesting that IFNκ is actively turned off during the differentiation process. This coincides with increased expression of the DNA methyltransferase DNMT3B (FIG. 3E), which is demonstrated to be responsible for the IFNK promoter hypermethylation in KO KCs, and suppression of IFNκ mRNA expression (FIG. 3C-D, FIG. 9). The data therefore show that CRISPR transfection is more efficient in cells where IFNκ has been “turned-off” through promoter methylation thereby selecting for those KCs. This is analogous to HPV infections where there is a selection for KCs that do not express IFNκ (32), and additionally provides an explanation for why HPV infections and viral replication predominantly involve mid to upper layers of the epidermis (29). In this context it is worth noting that DNMT3B expression has been shown to correlate with HPV infection (33,34), and furthermore, APOBEC3 members have been shown to act as restriction factors of HPV infection (35). HPV can also actively suppress IFNκ expression through the function of the oncogenic proteins E6 and E7 (36, 37). This likely enables the virus to gain entry into the lower layers of the epidermis where the epidermal stem cells reside.

Few studies have looked at immunological processes that may interfere with CRISPR transfection. One obstacle has been the potential immunogenicity of the Cas proteins, particularly regarding pre-existing adaptive immunity to Streptococcus pyogenesis and Staphylococcus aureus (38-40). In terms of cellular mechanisms, CRISPR gene editing is more efficient in cells that have lost the function of the tumor suppressor p53 in retinal epithelial cells (41) and in human pluripotent stem cells (42). The use of CRISPR or other gene therapy approaches to correct various inherited disorders of the skin hold great promise. Provided herein are systems and methods that facilitate such approaches.

In some embodiments, cells, whether in vitro (e.g., in culture), ex vivo, or in vivo are contacted with an agent (e.g., a JAK1 and/or JAK2 inhibitor) to increase efficiency of transfection and then are either simultaneously or subsequently transfected with a desired nucleic acid molecule (e.g., a CRISPR-Cas system nucleic acid molecule). Any agent that is capable of inhibition may be used. Inhibitors may be small molecules, antibodies, proteins, peptides, nucleic acid molecules, or the like. In some embodiments, two or more different inhibitors are contacted to the cells. Inhibitors may be formulated as desired for the nature of the cell being treated. For example, inhibitors may be in solution for use in cell culture and formulated for appropriate for the desired administration route for in vivo uses (e.g., for topical administration, for systemic administration).

In some embodiments, a compound, a derivative thereof, or a pharmaceutically acceptable salt thereof, is administered in a pharmaceutically effective amount. In some embodiments, a compound, a derivative thereof, or a pharmaceutically acceptable salt thereof, is administered in a therapeutically effective dose. The dosage amount and frequency are selected to create an effective level of the compound without substantially harmful effects. When administered topically, orally, or intravenously, the dosage of the compound or related compounds will generally range from 0.001 to 10,000 mg/kg/day or dose (e.g., 0.01 to 1000 mg/kg/day or dose; 0.1 to 100 mg/kg/day or dose).

Methods of administering a pharmaceutically effective amount include, without limitation, administration in parenteral, oral, intraperitoneal, intranasal, topical, sublingual, rectal, and vaginal forms. Parenteral routes of administration include, for example, subcutaneous, intravenous, intramuscular, intrasternal injection, and infusion routes. In some embodiments, the compound, a derivative thereof, or a pharmaceutically acceptable salt thereof, is administered orally.

In some embodiments, a single dose of a compound or a related compound is administered to a cell, tissue, or subject. In other embodiments, multiple doses are administered over two or more time points, separated by hours, days, weeks, etc. In some embodiments, compounds are administered over a long period of time (e.g., chronically), for example, for a period of months or years (e.g., 1, 2, 3, 4, 5, 6, 7, 8, 9, 10, 11, 12, or more months or years). In such embodiments, compounds may be taken on a regular scheduled basis (e.g., daily, weekly, etc.) for the duration of the extended period.

JAK (Janus kinase) inhibitors include JAK1 and JAK2 inhibitors as well as inhibitors of other members of the Janus kinase family of enzymes (e.g., JAK3, TYK2). Such inhibitor include, but are not limited to, Ruxolitinib (trade names Jakafi/Jakavi) (JAK1/JAK2), Tofacitinib (trade names Xeljanz/Jakvinus, formerly known as tasocitinib and CP-690550) (JAK3), Oclacitinib (trade name Apoquel) (JAK1), Baricitinib (trade name Olumiant) (JAK1/JAK2), Peficitinib (ASP015K, JNJ-54781532; trade name Smyraf) (JAK3), Fedratinib (SAR302503; trade name Inrebic) (JAK2), Upadacitinib (trade name Rinvoq; ABT-494) (JAK1), Filgotinib (G-146034, GLPG-0634) (JAK1), Cerdulatinib (PRT062070) (dual SYK/JAK), Gandotinib (LY-2784544) (JAK2), Lestaurtinib (CEP-701) (JAK2), Momelotinib (GS-0387, CYT-387) (JAK1/JAK2), Pacritinib (SB1518) (JAK2), Abrocitinib (PF-04965842) (JAK1), Deucravacitinib (BMS-986165) (TYK2), Ruxolitinib cream (INCB018424) (JAK1/2), Cucurbitacin I (JSI-124), CHZ868 (JAK2), Tofacitinib, and Topical tofacitinib and ruxolitinib, as well as compounds described in U.S. Pat. Nos. 8,609,687, 8,637,526, 8,987,443, 9,422,300, 9,890,165, 10,023,577, 10,100,049, 10,435,428, 10,493,077, 10,617,690, 10,758,543, 10,786,507, and 10,968,222 and US Pat. Pub. Nos. 20210155621, 20210030672, and 20200190080, each of which is herein incorporated by reference in its entirety.

In some embodiments, the nucleic acid that is transfected into the skin cells encodes a portion of or all of a CRISPR-Cas system (e.g., a CRISPR-Cas9 system). In some embodiments, the system encodes one or more Clustered Regularly Interspaced Short Palindromic Repeats associated (Cas) proteins (e.g., catalytically dead Cas9). In some embodiments, the system provides a guide RNA. The gRNA itself comprises a sequence complementary to one strand of the DNA target sequence and a scaffold sequence which binds and recruits Cas9 to the target DNA sequence.

The guide RNA (gRNA) may be a crRNA, crRNA/tracrRNA (or single guide RNA, sgRNA). The gRNA may be a non-naturally occurring gRNA. The terms “gRNA,” “guide RNA” and “guide sequence” may be used interchangeably throughout and refer to a nucleic acid comprising a sequence that determines the binding specificity of a Cas protein. A gRNA hybridizes to (complementary to, partially or completely) the DNA target sequence.

The gRNA or portion thereof that hybridizes to the target nucleic acid (a target site) may be any length necessary for selective hybridization. gRNAs or sgRNA(s) can be between about 5 and about 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

To facilitate gRNA design, many computational tools have been developed (See Prykhozhij et al. (PLoS ONE, 10(3): (2015)); Zhu et al. (PLoS ONE, 9(9) (2014)); Xiao et al. (Bioinformatics. January 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

While Cas9 systems have been very well characterized, there are many of CRISPR-Cas system or similar systems that have been identified and characterized. The compositions and methods described herein find use with any of these systems.

### Examples

### Keratinocyte Cell Culture:

Immortalized keratinocyte cell line, N/TERT1, was used with permission for generation of knock-out (KO) cell lines using non-homologous end joining via CRISPR/Cas9. This cell line has been shown to have normal differentiation characteristics in both monolayer and organotypic skin models (1). N/TERTs were grown in Keratinocyte-SFM medium (ThermoFisher #17005-042) supplemented with 30 μg/ml bovine pituitary extract, 0.2 ng/ml epidermal growth factor, and 0.3 mM calcium chloride (2).

### Generation of CRISPR KO Lines in N/TERTs KCs:

CRISPR KO KCs were generated as previously described (3). In brief, Single-guide RNA (sgRNA) target sequence was developed using a web interface for CRISPR design (https://portals.broadinstitute.org/gpp/public/analysis-tools/sgrna-design). Synthetic sgRNA target sequences were inserted into a cloning backbone, pSpCas9 (BB)-2A-GFP (PX458) (Addgene plasmid #48138) and then cloned into competent E. coli (ThermoFisher #C737303). Proper insertion was validated by Sanger sequencing. The plasmid with proper insertion was then transfected into an immortalized KC line (N/TERTs) using the TransfeX transfection kit (ATCC #ACS4005) in presence or absence of JAK1/JAK2 inhibitor, baricitinib. GFP positive single cells were plated and then expanded. Cells were then genotyped and analyzed by Sanger sequencing.

### Generation of Overexpressing KCs Lines:

DNMT1, DNMT3A and DNMT3B over expressing KCs were generated by lentiviral transductions of mammalian vector containing Myc-DDK-tagged human DNMT1 (NM_001130823, Origene), DNMT3A (NM_175629, Origene), and DNMT3B (NM_006892, Origene) respectively. HEK293T cells were used for viral packaging. Briefly, 10 μg of expression vector was mixed with equal concentration of packaging plasmid (TR30037, Origene) in 1 ml Opti-MEM medium (31985062, Invitrogen) and 30 μl turbofectin (TF8100, Origene), incubated at room temperature for 5 minutes. The obtained mixture was added to the HEK293T cells without dislodging the cells. Supernatants from infected HEK293T cells were harvested after 24 hours, filtered using 0.45 μm syringe filters, aliquoted and stored at −80 C or used immediately. 0.25×10⁶ N/TERTs KCs were plated a day before transduction in serum and antibiotics-free medium. The next day, the cells were transduced at a multiplicity of infection of 0.5 along with 8 μg/ml of polybrene (TR-1003-G, Sigma Aldrich). 24 hours post-transfection, media was replaced with complete growth media. Cells were passaged the following day and the media containing puromycin was used from then on. Puromycin concentration for KCs was determined by performing a drug-kill curve. An empty mammalian expression vector containing Myc-DDK tag only (PS100001, Origene) was used as negative control for transduction experiments. Un-transduced cells were also treated with puromycin to observe complete cell death. Once all the cells in control wells were killed, limited dilution was performed to obtain single cells which were expanded, and the clones were verified for over expression by western blotting.

RNA Extraction, qRT-PCR and RNA-Sequencing (4):

RNA extraction, qRT-PCR and RNA-Sequencing were performed following the protocol published earlier (3). RNAs were isolated from cell cultures using Qiagen RNeasy plus kit (Cat #74136). qRT-PCR was performed on a 7900HT Fast Real-time PCR system (Applied Biosystems) with TaqMan Universal PCR Master Mix (ThermoFisher Scientific). Libraries for RNA-seq were generated from polyadenylated RNA and sequenced at six libraries per lane on the Illumina Genome Analyzer IIx. We used STAR to align RNA-seq reads to the human genome, using annotations of GENCODE as gene model. HTSeq was used to quantify gene expression levels; normalization and differential expression analysis were performed by DESeq2.

Single Cell RNA-Sequencing from Human Skin:

Generation of single cell suspensions for single cell RNA-sequencing (scRNA-seq) was performed as follows from normal human epidermis. Samples were incubated overnight in 0.4% dispase (Life Technologies) in Hank's Balanced Saline Solution (Gibco) at 4° C. Epidermis and dermis were separated. Epidermis was digested in 0.25% Trypsin-EDTA (Gibco) with 10 U/mL DNase I (Thermo Scientific) for 1 hour at 37° C., quenched with FBS (Atlanta Biologicals), and strained through a 70 μM mesh. Dermis was minced, digested in 0.2% Collagenase II (Life Technologies) and 0.2% Collagenase V (Sigma) in plain medium for 1.5 hours at 37° C., and strained through a 70 μM mesh. Epidermal and dermal cells were recombined, and libraries were constructed by the University of Michigan Advanced Genomics Core on the 10× Chromium system. Libraries were then sequenced on the Illumina NovaSeq 6000 sequencer to generate 151-bp paired end reads. Data processing including quality control, read alignment, and gene quantification was conducted using the 10× Cell Ranger software. Seurat was used for normalization, data integration, and clustering analysis (5). Clustered cells were mapped to corresponding cell types by matching cell cluster gene signatures with putative cell-type specific markers.

Single Cell ATAC-Sequencing from Human Skin:

Four mm skin biopsies were obtained from palm/hip from healthy individual. Biopsies were then incubated in 0.4% dispase overnight in order to separate the epidermis and dermis. After the separation, epidermis was transferred to 0.25% Trypsin-EDTA+10 unit/mL DNase mixture and incubated at 37° C. for 1 hr. Epidermis mixture was then quenched with FBS and precipitated by centrifugation. Cell pellets were then resuspended in PBS+0.04% BSA. Cell numbers were counted at this step for future dilution calculation. The nuclei isolation protocol was carried as described by 10× Genomics. Of note, cells obtained from epidermis were incubated in lysis buffer on ice for 7 min to achieve best lysis efficacy. The cell lysis efficacy was determined by Countess II FL Automated Cell Counter. The single cell ATACseq library was prepared by Advanced Genomics Core at University of Michigan. 10,000 nuclei/sample and 25,000 reads/nuclei were targeted, and the libraries were sequenced using NovaSeq SP 100 cycle flow cell. The raw data was first processed by the Chromium Single cell ATAC Software Suite (10× Genomics), and then analyzed using the Signac package in R. Briefly, the single cell ATACseq data go through a series of analyses including quality control, dimension reduction, clustering and integration with previously annotated single cell RNAseq data. DNA accessibility profile was then visualized in different cell types and samples.

Accell siRNA Knock Down:

N/TERTs KCs were plated in 96 well plate (30,000 cells/well) and incubated at 37° C. with 5% CO₂ overnight. 100 μM accell siRNA (Dharmacon, APOBEC3A-E-017432-00-0005, APOBEC3B-E-017322-01-0005, APOBEC3G-E-013072-00-0005, APOBEC3H-E-019144-00-0005, DNASE1-E-016280-00-0005) was prepared in Ix siRNA buffer (Dharmacon #B-002000-UB-100). 1 μl of 100 μM siRNA was diluted with 100 μl accell delivery medium (Dharmacon #B-005000) for each well of 96 well plate. Growth medium was removed from the cells and 100 μl of the appropriate delivery mix with siRNA was added to each well and the plate was incubated at 37° C. with 5% CO₂. Accell Non-targeting Control siRNA (Dharmacon #D-001910-01-05) was used as a negative control. After 72 hours, cells were harvested for RNA preparation. RNA isolation and qRT-PCR were as above.

### 3-D Human Epidermal Tissue Cultures:

Normal human epidermal KCs were isolated from a pool of neonatal foreskins (n=3) and grown using a J2-3T3 mouse fibroblasts as feeder layer as originally described by Rheinwald and Green (6). 3-D human epidermal raft cultures seeded in collagen hydrogels were prepared using three distinct donor pools as described previously (7) and grown at air-liquid interface for 12 days in E-Medium (DMEM/DMEM-F12 (1:1), 5% Fetal Bovine Serum, adenine (180 μM), Bovine pancreatic insulin (5 μg/ml), Human apo-transferrin (5 μg/ml), triiodothyronine (5 μg/ml), L-Glutamine (4 mM), Cholera toxin (10 ng/ml), Gentamicin (10 μg/ml), Amphotericin B (0.25 μg/ml)). After 9 days at an air-liquid-interface to allow for epidermal maturation, the RHEs were treated with 0.1% BSA/phosphate-buffered saline (Sigma Aldrich, St Louis, MO) as a vehicle control or 10.0 ng/ml TNF-α, IL-17A, IL-22 (R&D Systems, Minneapolis, MN) alone or as a combination for 72 h, harvested, and analyzed for changes in gene expression as described (8). Epidermal tissues were separated from the collagen scaffold and lysed in QIAzol for RNA isolation. RNA-seq and analysis were performed according methods mentioned above.

### Measurement of CRISPR Plasmids Stability in KCs:

CRISPR plasmid (PX458, Addgene #48138) was transfected into KCs using Transfex transfection kit (ATCC #ACS-4005). Cells were then harvested at different time points (0 Hr, 6 Hrs, 12 Hrs, 24 Hrs and 48 Hrs) and washed with PBS three times to remove extracellular plasmid from the cells. Then DNA was then purified using the QIAamp DNA Blood Mini kit (Qiagen #51106). CRISPR plasmid specific primers (Px458-F: GGGCAGAGGAAGTCTGCTAA (SEQ ID NO: 1) and Px458-R: GGGAGGGGCAAACAACAGAT (SEQ ID NO: 2)) were used to perform qPCR with the DNAs isolated from the transfected KCs using SYBR Green PCR Master Mix (ThermoFisher #4309155) on the 7300 Real-time PCR system (Applied Biosystems).

### Bisulfite Sequencing Analysis of the IFNK Promoter:

Bisulfite treatment was performed on DNA isolated from wild type and CRISPR knock-out KCs using the EZ DNA methylation-Gold kit (Zymo Research #D5005) according to the vendor's recommendations. Bisulfite converted DNA was amplified with the following primers, IFNK-BS583 F9: TGTTGGGATGGATTATTTAGGTATT (SEQ ID NO: 3) and IFNK-BS-R9: TTCAACAAAAAAAATTTTCTCATTC (SEQ ID NO: 4). PCR products were cloned in pCR2.1-TOPO vector (ThermoFisher #K204040) and those clones were then subjected to sanger sequencing using M13Rev and T7 primers.

### Western Blot

Total protein was isolated from cells using Pierce RIPA buffer (89900, ThermoFisher) with PMSF Protease Inhibitor (36978, Sigma) and run-on pre-cast gel (456-1094S, Bio-Rad). The membrane was blocked with 3% BSA and then probed by primary antibodies including p-IRF3 (ThermoFisher #PA536775), DNMT3B (Cell signaling #67259S) and β-Actin (A5441, Sigma), followed by secondary antibodies (anti-mouse or rabbit IgG, AP-linked Antibody, Cell Signaling), then washed for 3 times, and substrate added (45-000-947, Fisher Scientific). They were then imaged with (brand) chemiluminescent kit and imaged on iBright imager (ThermoFisher).

### Immunostaining:

Formalin-fixed, paraffin-embedded tissue slides obtained from healthy individual were heated for 30 min at 60° C., rehydrated, and epitope retrieved with tris-EDTA (pH 6). Slides were blocked and incubated with primary antibodies against IFN-κ (Abnova #H00056832-M01), APOBEC3G (Abcam #Ab223704) and DNMT3B (Cell Signaling #67259S) overnight at 4° C. Slides were incubated with biotinylated secondary antibodies (biotinylated goat anti-rabbit IgG antibody, BA1000, Vector Laboratories; biotinylated horse anti-mouse IgG antibody, BA2000, Vector Laboratories) and then incubated with fluorochrome-conjugated streptavidin. Slides were prepared in mounting medium with 4′,6-diamidino-2-phenylindole (DAPI) (VECTASHIELD Antifade Mounting Medium with DAPI, H-1200, VECTOR). Images were acquired using inverted Zeiss microscope. Images presented are representative of at least three biologic replicates.

### REFERENCES

1 Wu, S. S., Li, Q. C., Yin, C. Q., Xue, W. & Song, C. Q. Advances in CRISPR/Cas-based Gene Therapy in Human Genetic Diseases. Theranostics 10, 4374-4382, doi:10.7150/thno.43360 (2020).
2 Kocher, T. et al. Predictable CRISPR/Cas9-Mediated COL7A1 Reframing for Dystrophic Epidermolysis Bullosa. J Invest Dermatol, doi:10.1016/j.jid.2020.02.012 (2020).
3 Pickar-Oliver, A. & Gersbach, C. A. The next generation of CRISPR-Cas technologies and applications. Nat Rev Mol Cell Biol 20, 490-507, doi:10.1038/s41580-019-0131-5 (2019).
4 Suzuki, K. et al. In vivo genome editing via CRISPR/Cas9 mediated homology independent targeted integration. Nature 540, 144-149, doi:10.1038/nature20565 (2016).
5 Young, C. S. et al. A Single CRISPR-Cas9 Deletion Strategy that Targets the Majority of DMD Patients Restores Dystrophin Function in hiPSC-Derived Muscle Cells. Cell Stem Cell 18, 533-540, doi:10.1016/j.stem.2016.01.021 (2016).
6 Bilousova, G. Gene Therapy for Skin Fragility Diseases: The New Generation. J Invest Dermatol 139, 1634-1637, doi:10.1016fj.jid.2019.04.001 (2019).
7 Kocher, T. et al. Cut and Paste: Efficient Homology-Directed Repair of a Dominant Negative KRT14 Mutation via CRISPR/Cas9 Nickases. Mol Ther 25, 2585-2598, doi:10.1016/j.ymthe.2017.08.015 (2017).
8 Benati, D. et al. CRISPR/Cas9-Mediated In Situ Correction of LAMB3 Gene in Keratinocytes Derived from a Junctional Epidermolysis Bullosa Patient. Mol Ther 26, 2592-2603, doi:10.1016/j.ymthe.2018.07.024 (2018).
9 Wu, W. et al. Efficient in vivo gene editing using ribonucleoproteins in skin stem cells of recessive dystrophic epidermolysis bullosa mouse model. Proc Natl Acad Sci USA 114, 1660-1665, doi:10.1073/pnas.1614775114 (2017).
10 Maurisse, R. et al. Comparative transfection of DNA into primary and transformed mammalian cells from different lineages. BMC Biotechnol 10, 9, doi:10.1186/1472-6750-10-9 (2010).
11 Wang, J. et al. Transfection Efficiency Evaluation and Endocytosis Exploration of Different Polymer Condensed Agents. DNA Cell Biol 38, 1048-1055, doi:10.1089/dna.2018.4464 (2019).
12 Gresch, O. & Altrogge, L. Transfection of difficult-to-transfect primary mammalian cells. Methods Mol Biol 801, 65-74, doi:10.1007/978-1-61779-352-3_5 (2012).
13 Zare, S. et al. Isolation, cultivation and transfection of human keratinocytes. Cell Biol Int 38, 444-451, doi:10.1002/cbin.10218 (2014).
14 Jiang, R. et al. Single-cell repertoire tracing identifies rituximab-resistant B cells during myasthenia gravis relapses. JCI Insight 5, doi:10.1172/jci.insight.136471 (2020).
15 Abe, T. & Barber, G. N. Cytosolic-DNA-mediated, STING-dependent proinflammatory gene induction necessitates canonical NF-kappaB activation through TBK1. J Virol 88, 5328-5341, doi:10.1128/JVI.00037-14 (2014).
16 Tsuchiya, Y., Jounai, N., Takeshita, F., Ishii, K. J. & Mizuguchi, K. Ligand-induced Ordering of the C-terminal Tail Primes STING for Phosphorylation by TBK1. EBioMedicine 9, 87-96, doi:10.1016/j.ebiom.2016.05.039 (2016).
17 LaFleur, D. W. et al. Interferon-kappa, a novel type I interferon expressed in human keratinocytes. J Biol Chem 276, 39765-39771, doi:10.1074/jbc.M102502200 (2001).
18 Li, Y. et al. Interferon Kappa Is Important for Keratinocyte Host Defense against Herpes Simplex Virus-1. Journal of Immunology Research 2020, 1-8, doi:10.1155/2020/5084682 (2020).
19 Doorbar, J., Egawa, N., Griffin, H., Kranjec, C. & Murakami, I. Human papillomavirus molecular biology and disease association. Rev Med Virol 25 Suppl 1, 2-23, doi:10.1002/rmv.1822 (2015).
20 Stenglein, M. D., Burns, M. B., Li, M., Lengyel, J. & Harris, R. S. APOBEC3 proteins mediate the clearance of foreign DNA from human cells. Nat Struct Mol Biol 17, 222-229, doi:10.1038/nsmb.1744 (2010).
21 Kawane, K., Motani, K. & Nagata, S. DNA degradation and its defects. Cold Spring Harb Perspect Biol 6, doi:10.1101/cshperspect.a016394 (2014).
22 Oliveri, M. et al. DNase I mediates internucleosomal DNA degradation in human cells undergoing drug-induced apoptosis. European Journal of Immunology 31, 743-751, doi:10.1002/1521-4141(200103)31:3<743::Aid-immu743>3.0.Co; 2-9 (2001).
23 Jang, H. S., Shin, W. J., Lee, J. E. & Do, J. T. CpG and Non-CpG Methylation in Epigenetic Gene Regulation and Brain Function. Genes (Basel) 8, doi:10.3390/genes8060148 (2017).
24 Edwards, J. R., Yarychkivska, O., Boulard, M. & Bestor, T. H. DNA methylation and DNA methyltransferases. Epigenetics Chromatin 10, 23, doi:10.1186/s13072-017-0130-8 (2017).
25 Moulin, L. et al. Methylation of LOXL1 Promoter by DNMT3A in Aged Human Skin Fibroblasts. Rejuvenation Res 20, 103-110, doi:10.1089/rej.2016.1832 (2017).
26 Wikramanayake, T. C., Stojadinovic, O. & Tomic-Canic, M. Epidermal Differentiation in Barrier Maintenance and Wound Healing. Adv Wound Care (New Rochelle) 3, 272-280, doi:10.1089/wound.2013.0503 (2014).
27 Jiang, Y. et al. Cytokinocytes: the diverse contribution of keratinocytes to immune responses in skin. JCI Insight 5, doi:10.1172/jci.insight.142067 (2020).
28 Sarkar, M. K. et al. Photosensitivity and type I IFN responses in cutaneous lupus are driven by epidermal-derived interferon kappa. Ann Rheum Dis 77, 1653-1664, doi:10.1136/annrheumdis-2018-213197 (2018).
29 Egawa, N., Egawa, K., Griffin, H. & Doorbar, J. Human Papillomaviruses; Epithelial Tropisms, and the Development of Neoplasia. Viruses 7, 3863-3890, doi:10.3390/v7072802 (2015).
30 Doorbar, J. Molecular biology of human papillomavirus infection and cervical cancer. Clin Sci (Lond) 110, 525-541, doi:10.1042/CS20050369 (2006).
31 Lau, L., Gray, E. E., Brunette, R. L. & Stetson, D. B. DNA tumor virus oncogenes antagonize the cGAS-STING DNA-sensing pathway. Science 350, 568-571, doi:10.1126/science.aab3291 (2015).
32 DeCarlo, C. A. et al. IFN-kappa, a novel type I IFN, is undetectable in HPV-positive human cervical keratinocytes. Lab Invest 90, 1482-1491, doi:10.1038/labinvest.2010.95 (2010).
33 Leonard, S. M. et al. Oncogenic human papillomavirus imposes an instructive pattern of DNA methylation changes which parallel the natural history of cervical HPV infection in young women. Carcinogenesis 33, 1286-1293, doi:10.1093/carcin/bgs157 (2012).
34 Lin, T. S. et al. An association of DNMT3b protein expression with P16INK4a promoter hypermethylation in non-smoking female lung cancer with human papillomavirus infection. Cancer Lett 226, 77-84, doi:10.1016/j.canlet.2004.12.031 (2005).
35 Warren, C. J. et al. APOBEC3A functions as a restriction factor of human papillomavirus. J Virol 89, 688-702, doi:10.1128/JVI.02383-14 (2015).
36 Rincon-Orozco, B. et al. Epigenetic silencing of interferon-kappa in human papillomavirus type 16-positive cells. Cancer Res 69, 8718-8725, doi:10.1158/0008-5472.CAN-09-0550 (2009).
37 Reiser, J. et al. High-risk human papillomaviruses repress constitutive kappa interferon transcription via E6 to prevent pathogen recognition receptor and antiviral-gene expression. J Virol 85, 11372-11380, doi:10.1128/JVI.05279-11 (2011).
38 Simhadri, V. L. et al. Prevalence of Pre-existing Antibodies to CRISPR-Associated Nuclease Cas9 in the USA Population. Mol Ther Methods Clin Dev 10, 105-112, doi:10.1016/j.omtm.2018.06.006 (2018).
39 Wagner, D. L. et al. High prevalence of Streptococcus pyogenes Cas9-reactive T cells within the adult human population. Nat Med 25, 242-248, doi:10.1038/s41591-018-0204-6 (2019).
40 Charlesworth, C. T. et al. Identification of preexisting adaptive immunity to Cas9 proteins in humans. Nat Med 25, 249-254, doi:10.1038/s41591-018-0326-x (2019).
41 Haapaniemi, E., Botla, S., Persson, J., Schmierer, B. & Taipale, J. CRISPR-Cas9 genome editing induces a p53-mediated DNA damage response. Nat Med 24, 927-930, 345 doi:10.1038/s41591-018-0049-z (2018).
42 Ihry, R. J. et al. p53 inhibits CRISPR-Cas9 engineering in human pluripotent stem cells. Nat Med 24, 939-946, doi:10.1038/s41591-018-0050-6 (2018).

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A composition comprising:
a) one or more JAK1 and/or JAK2 inhibitors;
b) one or more transfection reagents; and
c) one or more nucleic acid molecules.

2. The composition of claim 1, wherein said one or more nucleic acid molecules comprises a vector.

3. The composition of claim 2, wherein said vector expresses one or more components of a CRISPR-Cas system.

4. The composition of claim 1, wherein said one or more nucleic acid molecules comprises a guide sequence.

5. The composition of claim 2, wherein said vector expresses one or more transgenes.

6. The composition of claim 4, said one or more transgenes comprises a gene involved in regulating a skin cell disease or condition.

7. The composition of claim 1, further comprising one or more skin cells.

8. The composition of claim 7, wherein said one or more skin cells comprises a keratinocyte.

9. Use of a composition of any of claims 1-8.

10. Use of a composition of any of claims 1-8 for transfecting a skin cell.

11. A method comprising:
a) contacting a skin cell with a JAK1 and/or JAK2 inhibitor; and
b) transfecting the skin cell with one or more nucleic acid molecules.

12. The method of claim 11, wherein said one or more nucleic acid molecules comprises a vector.

13. The method of claim 12, wherein said vector expresses one or more components of a CRISPR-Cas system.

14. The method of claim 11, wherein said one or more nucleic acid molecules comprises a guide sequence.

15. The method of claim 12, wherein said vector expresses one or more transgenes.

16. The method of claim 14, said one or more transgenes comprises a gene involved in regulating a skin cell disease or condition.

17. The method of claim 11, wherein the skin cell is in vivo.

18. The method of claim 11, wherein the skins cell is ex vivo.

19. The method of claim 11, wherein the skin cell is in vitro.

20. The method of claim 11, wherein the skin cell comprises a keratinocyte.


SEQUENCE LISTING (4 sequences)

SEQ ID NO: 1 - DNA, 20 bp, Artificial sequence
  synthetic
gggcagaggaagtctgctaa

SEQ ID NO: 2 - DNA, 20 bp, Artificial sequence
  synthetic
gggaggggcaaacaacagat

SEQ ID NO: 3 - DNA, 25 bp, Artificial sequence
  synthetic
tgttgggatggattatttaggtatt

SEQ ID NO: 4 - DNA, 25 bp, Artificial sequence
  synthetic
ttcaacaaaaaaaattttctcattc